(19) United States
(12) Patent Application Publication
(10) Pub. No.: US 2011/0313666 A1
(43) Pub. Date: Dec. 22, 2011

(54) METHOD FOR MONITORING ENVIRONMENTAL IMPACTS OF CROP CULTIVATION

(75) Inventors: Tero Hirvi, Turku (FI); Jaakko Laurinen, Turku (FI); Pasi Lähdetie, Muurla (FI)

(73) Assignee: RAISIO NUTRITION LTD, Raisio (FI)

(21) Appl. No.: 13/057,949

(22) PCT Filed: Aug. 7, 2009
(86) PCT No.: PCT/FI2009/050650

§ 371 Date: May 6, 2011

(30) Foreign Application Priority Data
Aug. 8, 2008 (FI) 20085764

(51) Int. Cl. G06F 19/00

(52) U.S. Cl. 702/2

(57) ABSTRACT

The invention relates to a method, a system, apparatus, and software to be used for the method for the determination of the environmental impact of the production of cultivation plants and for increasing or maximizing a positive environmental impact. In the method, production parameters for cultivation are selected and cultivation procedures are performed, crop yieldhus produced is harvested, and a representative sample of the crop yield is delivered to reception analysis with the appendant information for the analysis of energy and carbon dioxide factors. An environmental impact index reflecting the environmental impact of the production of said cultivation plant is determined on the basis of this data and the appendant information. The index thus provided is utilized for the production of cultivation plants in an environmentally friendlier manner.

The invention relates to a method for monitoring the environmental impact of agricultural production, and for controlling said impact by means of balance computations of energy and carbon dioxide required in crop production, and measures indicated as necessary by said computation.

### BACKGROUND

In crop production, emissions, for instance carbon dioxide emissions released in the environment may be controlled by suitable selection of environmentally friendly cultivation procedures. As is known, the amount of energy consumed and produced in industrial processes per production volumes is monitored, for instance for energy production by power plants, such monitoring being also performed for instance to assess energy consumption for one kg of a fertilizer produced and emissions from the production, or more specifically, to assess the impact of feed on the productivity of dairy cows in animal husbandry, or the impact of energy contained in different feed products on growth rate of meat producing piglets, and unutilized minerals found in animal manure. It is also possible to control emissions and energy consumption in crop production.

By application of balance computation models, for instance the carbon dioxide balance of oat flakes was determined, showing that the amount of carbon dioxide bound from the atmosphere in the production of biomass is higher than that released during processing, that is, the carbon dioxide balance is negative by 460 g. So far, there are only few analyses assessing carbon dioxide emissions for food products, particularly for primary production thereof, said analyses being only carried out to determine the impact of processing in food chain.

In the future, an aim could be to provide packages of food products with indications of carbon dioxide emissions or energetic environmental impacts caused by the production thereof in addition to nutritional values declared at present. Thus, consumers could acquire desired information about the environmental impacts of the production of a particular food product and also raw materials therefor, to allow comparison of different food products with one another, and to make use of said information for purchase decision. Thus, each consumer may with a personal choice make a difference and knowingly help to increase the use of products with favourable environmental impacts. In the consumption of the Finns, considerable proportion, about one third, of carbon dioxide emissions is caused by food, the balance by housing and traffic.

Farmers can hardly influence inherent properties of soil such as soil type, thickness of fertile layer, or pore structure, or release rates of nutrients, oxides of nitrogen or carbon dioxide due to said properties. Moreover, only limited influence on climatic factors like precipitation is possible by farmers, an example being combatting dryness by artificial irrigation.

In agriculture, balance computation for instance for the general quantitative determination of nutrients has previously been utilized. These computations typically focus on nutrient balance with a computation method for monitoring nutrient flows in agriculture. There are several kinds of nutrient balances, such as field balance, gate balance, livestock balance, and manure balance.

Calculation of the difference between nutrient amounts added to and removed from fields, the so-called field balance, indicates the degree of utilization of the nutrients in fertilizers. Typically, utilization of nitrogen and phosphorus fertilizers is of interest. In case more nutrients are added to the field than removed therefrom with the crop, the risk of nutrient washout increases and economic feasibility of cultivation decreases.

In the calculation of the gate balance, amounts of nutrients in fertilizers and animal matter, and other production inputs introduced to the farm are determined. The amounts of nutrients leaving the farm with crop or other products are then subtracted from the introduced amounts. The result is the so-called gate balance indicating the total degree of utilization of nutrients on the farm. Calculation of the gate balance is reasonably carried out for a calendar or crop year.

Livestock balance shows the efficiency of utilization of feed nutrients, and the proportion thereof excreted in manure. Nutrients are acquired by livestock with feed purchased for the farm, and leave the livestock in the form of animal products and sold animals.

Manure balance is the difference between the nutrients excreted from the animals in manure, and delivered to field from the manure. The proportion of the nutrients of manure unutilized for fertilization may be determined by subtracting the amount of nutrients in manure applied on the field from the livestock balance.

One known way to discuss various steps of production as a whole is its life cycle analysis (LCA). According to the definition, LCA is a study for analyzing and assessing environmental impacts of a product. In the study, material and energy consumptions, emissions to the atmosphere and surface waters, as well as the amount of solid waste during the whole life cycle of the product are taken into consideration. Typically, the life cycle of a product begins with purchase of raw materials and required energy, and covers the production, distribution and use of the product. Further, recycling of used product and removal from the circulation may also be taken into consideration.

One aim of the Finnish Environment Centre is the comparison of the environmental impact of traditional agricultural production to that of organic farming. In several studies, amounts of energy to produce production inputs and CO₂ emissions therefrom are collected. For instance, most significant emissions and residues of the whole life cycle of milk and rye bread may be calculated for an operational unit. 1000 litres of milk on a store shelf and 1000 grams of rye bread on a store shelf serve as operational units. Such an assessment makes it difficult to compare and estimate environmental impacts of different product groups with each other when for instance milk and rye bread are compared with each other. Comparison is possible only between similar or equal products. No data about substantial differences between emissions of different production methods is presented.

Alternative working steps of the production chain, energy consumption thereof and energy values are described in various studies of the Technical Research Centre of Finland (VTT). The amount of energy and emissions for the production of different production inputs have been estimated with respect to carbon dioxide and oxides of nitrogen. As target plants, for instance barley for ethanol production, rapeseed and Phalaris arundinacea (reed canarygrass) for biodiesel production and similar sources from forestry have been used. Processed ethanol or diesel, that is the final product, serves as the operational unit. Here, the discussion only relates to the life cycle analysis (LCA) for these products.

The patent publication WO 2006135880 presents method and software for selecting seed products or the like for sowing (drilling within a target site, including classifying the target site with an environmental classification, determining at least one seed product to be sown (drilling within the target site based on the environmental classification, and providing an output comprising identification of the at least one seed product for drilling within target site. Said method may contain specific cultivation data or other geospatial reference information. Also economic aspects may be associated with the selection of seed products or the like. The document relates to a risk analysis for the selection of proper crop seeds according to the environmental profile of the cultivation area by utilizing applicability data from the databank. Neither the evaluation of cultivation operations for energy and carbon dioxide nor the comparison of environmental impacts of various plants is possible with the method.

The patent publication WO2008070792 describes a method for utilization of reduced greenhouse emissions in electronic emission trading. Nitrogen amount for the provision of a genetically engineered crop is lower due to higher nitrogen efficiency of these plants than for non-engineered plants, resulting in reduced computational emission impacts on the atmosphere. This emission deficit may be commercially profited. However the reduction of emissions by means of cultivation procedures, or the implementation of this reduction is not described.

The publication Koga, N., An energy balance under a conventional crop rotation system in northern Japan: Perspectives on fuel ethanol production from sugar beet, Agriculture, Ecosystems and Environment 125 (2008) 101-110, described a computation method utilizing energy balances. In the method, precise calorimetric energy determinations are theoretically calculated using energetic feasibility of biomass production for bioethanol applications. Autumn wheat, sugar beet, adzuki-beans and potato were used as energy sources. In the balance calculations, also energy impacts of soil tilling and working procedures were taken into consideration. Carbon dioxide impact of cultivation, or other environmental impacts due to energy production were not of interest.

The object of the present invention is to enable a single farmer to take into consideration, assess and develop the amount of energy consumed and emissions released in the production of crop plants, and to control or modify the environmental impact associated with the production on the respective farm.

Another object of the invention is to provide a farmer with a less complicated method for the assessment of environmental impacts due to the agricultural production, and for the modification thereof in an environmentally favourable direction.

### SUMMARY OF THE INVENTION

The present invention provides a method for determining the environmental impacts of the production of crop plants, and for increasing or maximizing a positive environmental impact, as defined in claim 1. Moreover, the invention provides an environmental impact index obtainable with the method, according to claim 8, and the use of said index. Further, the invention provides a system to be used for the method, according to claim 10, whereas an apparatus is claimed claim 11, and software to be used in the systems is claimed in claim 12.

The invention provides farmers with a simple way to determine the influence of preselected production parameters and cultivation procedures on the environmental impact of crop production, such as climatic impact, and with a possibility to control a numerical value reflecting this environmental impact by means of their own choices and measures resulting from these choices.

Moreover, the awareness of consumers of the environmental impact of the primary production for the food products chosen may be increased in a simple and well understood manner. At the same time, consumers may be provided with the possibility to alter their consuming practices to a direction that is environmentally more friendly, consistent with the principle of sustainable development.

According to a further aspect of the invention, the environmental impacts of the production of various plants may be converted to be commensurate, thus making the comparison of easier. For instance for oil plants such as rape seed, seed yield is typically about 2000 kg of dry matter per hectare, while for starch plants such as cereals, seed yield is about 3000 kg of dry matter per hectare. Conversion of the harvested dry matter of both plant species into energy content shows that said energy content is about 48 GJ/hectare for both plants.

### DETAILED DESCRIPTION OF THE INVENTION

The method according to the first aspect of the invention assists the farmer to determine in a simple way and with reasonable accuracy the environmental impact of the cultivation of the crop plants the farmer produces. Once the environmental impact is known, it may be influenced by the choices of the farmer and measures resulting therefrom. By means of these choices, the production may be developed and thus the environmental impact due to said production may be altered, thus improving or maximizing the final positive environmental impact. Assessment of the environmental impact is partly based on one hand on the determination of the energy consumed by the plant in question during its life cycle, that is, its share in the input, and on the other hand, the energy produced by or bound in the plant, that is, its share in the output. Moreover, the amount of carbon dioxide bound by the plant, and the carbon dioxide emissions produced during cultivation are added to said input and output values. Further, nutrient balance may also be taken into consideration in the calculations, said nutrient balance measuring the amounts of nutrients removed with the crop, and used for the production. A positive nutrient balance reflects nutrient amounts liable to be washed out in kilograms per hectare. In addition to CO₂ emissions, other emissions such as those of nitrous oxide (N₂O), ammonia (NH₃) and methane (CH₄) released directly from the production to the atmosphere may also be taken into consideration in the method of the invention, according to currently valid regulations of IPCC (Intergovernmental Panel on Climate Change). These other emissions may be converted into CO₂ equivalence, CO₂e, according to established rules.

In this invention, a positive environmental impact means an environmental impact where the output in the crop produced by cultivating a crop plant is higher than the input used therefor, that is the object is to maximize the ratio of output to input.

Data preselected by the farmer in question based on his own cultivation scheme are required as the starting information. These data are typically specific for the farmer, and consist of a set of several parameters repeating from one cultivation season to another, and most applicable values may be selected for said parameters according to circumstances and expectations of the farmer. Cultivation procedures carried out on the basis of choices of the farmer are documented for the determination of the energy and carbon dioxide footprints of the production using storage, computation, analysis and printing systems of the invention to obtain so-called appendant information.

According to an embodiment of the invention, production parameters for the cultivation are selected and cultivation procedures are carried out by using an assumed input that is as low as possible with respect to assumed output. Production parameters used for the cultivation, and cultivation procedures determined by said parameters are selected on the basis of experience, know-how and yield-expectations of the farmer. Energy consumption of individual operations based on the selected parameters, and emissions of greenhouse gases or carbon dioxide equivalents, CO₂e, preferably carbon dioxide are measured, estimated or calculated in a known manner. Thus for each production parameter or cultivation operation, its share in total energy consumption may be determined. Further, carbon dioxide emissions (kg of CO₂/ton) for each production parameter or cultivation operation may be assessed. Net sum of these factors, the so-called environmental impact index, gives the total environmental impact. The object is to select the production parameters for the cultivation carried out by a specific farmer for increasing or maximizing the positive environmental impact, and to carry out the selected or necessary cultivation operations by using, with respect to the energy output from the crop for instance in the form of seeds, a low expected energy input for the provision of a crop.

The same production input is not necessarily associated with a similar or, relatively speaking, equal energy consumption or carbon dioxide emissions in comparison to other production inputs or methods. The study has typically revealed that for instance the energy consumption of plant protecting agents may be very low, as calculated for field hectare, while CO₂ emissions caused by the same production input may be very high, thus making it difficult to assess the total impact.

Production parameters of the cultivation and cultivation procedures to be selected preferably comprise information on soil tilling, more preferably information on soil tilling procedures and dates; information on fertilization, more preferably information on fertilizers used, used amounts of fertilizers and frequency of application; information on the use of machines and apparatuses, more preferably information on fuel consumption for said machines and times of use; information on plant protection, more preferably information on used agents, amounts and frequency of applications thereof; and harvesting information, more preferably information on the crop quality and amount, ways of harvesting, subsequent treatments, transportation distances and means of transportation.

Soil tilling may consist of traditional ploughing—harrowing—sowing, or reduced tilling of stubble field—direct drilling according to the crop, soil type of the field and date of tilling. In some cases, direct drilling is the best choice for soil tilling purchased as freight work and sowing, however, possibly resulting in lower yield than for fields cultivated by ploughing. Selection of the soil tilling method largely depends on the soil type of the field and soil moisture conditions. Reduced tilling procedures and retaining the stubble field over the winter decrease the nutrient leaching On the other hand, efficient tilling methods eradicate plant diseases and reduce weeds, thus decreasing the need for pesticides The less driving is needed and the lower the load of tractors caused by tilling procedures is, the lower is the total energy consumption of tilling procedures for hectare.

Quality of the selected fertilizer, used amounts thereof and the timing of fertilizing influence the amount of energy required for the use of fertilizers and nutrient balance. Energy input in the production of fertilizers is determined by the quality of fertilizers. For instance in the production of ammonium nitrate, effective nitrogen is provided in a considerably more economical manner with respect to energy consumption than for fertilizers containing multiple nutrients. It is important to select qualities with the highest activities, amounts, and application procedures for the plant crop. For instance, the efficiency of phosphorus may be significantly improved by application thereof in the vicinity of the seed, in the same row with the seeds. Application amounts of phosphorus may thus be reduced. On the other hand, use of technology may increase loads caused by cultivation units, and accordingly energy consumed. In addition, necessary energy used by machines for the application of fertilizers must be taken into consideration in the form of fertilization times and amounts. For instance, application of nitrogen and potassium may be timed prior to actual sowing, and for said application, surface application apparatuses consuming little energy and having high application efficiencies may be used instead of simultaneuos sowing and fertilization.

Seeds to be sown may comprise purchased certified seeds treated with pesticides or farm saved seeds from crop produced on the respective farm in previous year. Pesticides used for the treatment may contain e.g. triadienol, imazalil, carboxin as active agents. In addition to agents used for seed treatment, herbicides for weed control may be used when the crop is established It may also be necessary to control plant diseases such as fungal infections or diseases causing spots, or to prevent the crop plants from lodging by using growth regulators or to use fungicides.

Energetic impacts of said operations are determined by the quality and amounts of agents used, art of usage and way of application to the field.

Use of machines and apparatuses is determined by selected ways of soil tilling, fertilization, sowing, harvesting and/or pesticide uses Energy consumption for each machine or apparatus may be for instance derived from the engine power, duration of use and driving speed, said energy consumption being recorded for each cultivation operation. Composition of the whole machinery of a farm has a substantial influence on energy consumption thereof.

Once the crop is ripe, it is generally harvested by threshing, and thus it is necessary for instance to take the used thresher and the way of threshing into consideration. Moreover, transportation of the crop from the fields to subsequent treatments, such as into hot air dryers, or to straw baling demands transportation, treatment and drying equipment capacity and consumes energy. Further, working energy is consumed by transportation and treatment equipment for crop storage, such as introduction to silos, and for further processing in mills.

In one embodiment of the invention, the cultivated plant is selected from the group consisting of oil plants, preferably turnip rape, oil seed rape, Camelina, sunflower or soybean; cereals for human and animal nutrition, preferably wheat, rye, barley or oat; leguminous plants, preferably peas or broad bean; potato, corn and sugar beet.

Once the crop is ready for harvesting, the part of the crop with the highest energy content, so-called crop yield, typically comprising the seeds, tubers, pods or other useful plant parts, is harvested for further processing. Preferably for the energy balance, secondary crops that is, side crops with less value for energy production and further processing such as straws and/or roots or another crop byproducts containing energy, useful for instance to produce heat for the farm, may also be incorporated into the crop yield. Part of the crop yield is separated from the crop yield to obtain a qualitatively representative working sample, such as representative seed sample, and this sample is passed to reception analysis together with appendant information. This reception analysis may be carried out in any laboratory capable of analysing the qualitative factors of the crop in question with significance for further processing. The reception analysis is preferably carried out in a laboratory of a plant for further processing, such as cereal grain laboratory.

The term appendant information refers to cultivation procedures described above and selected by an individual farmer, for which energy and carbon dioxide factors, and possibly the nutrient balance may be determined. Appendant information reflecting the cultivation procedures carried out by the farmer may be converted into energy and carbon dioxide factors in a known manner. For instance, the constant energy value of fuel oil consists of heat value, 43 MJ/kg, and carbon dioxide emission, 2.7 kg CO₂/l. Energy used by machines for cultivation operations may be calculated from utilization and consumption data of the machine. Also estimates for the energy consumption (GJ) of machines per cultivated hectare are available. Appendant information preferably contains a set of parameters comprising: information on soil tilling, information on fertilizers used, information on the use of machines and apparatuses, information on plant protection and harvesting information. The appendant information is supplied with the representative working sample of the crop. The appendant information is preferably in a recorded form, such as a prefilled form, more preferably appended with the representative working sample of the crop yield. Most preferably, the prefilled form is attached to a container for the representative sample, such as a sample bag filled e.g. with a seed sample, or the form is an integral part of the bag, obviating separate information forms. Still more preferably, the information is stored on a microchip containing the information of the form.

From the representative working sample, energy and carbon dioxide factors of the crop yield are analysed in the reception analysis.

The term energy factors of the crop yield refers to qualitative factors typical for the crop that may be analyzed and converted into energy values. In case of cereals, these energy factors preferably comprise the amounts of starch, protein and fat, ash content and qualitative features relevant to technical utility, preferably falling number, hectoliter weight, and amounts of wastes and residues. As is known, constant values for energy factors have been determined. For instance, the energy values for one gram of starch, for one gram of protein, and for one gram of plant fat are 1.74 kJ, 2.34 kJ and 3.68 kJ, respectively.

The term carbon dioxide factor refers to all qualitative properties of a crop that may be analyzed and converted into bound carbon dioxide. As is known, the amount of carbon dioxide bound to the crop may be assessed based on the amount of the crop since it is known that the amount of bound carbon dioxide to produce one kilogram of dry matter is constant for any dry matter, being e.g. 1.47 kg CO₂/kg of dry matter for wheat.

Technical utility is reflected by measurable properties, and for instance for cereals, the application of the crop, and accordingly further processing thereof is preferably determined by the falling number, hectoliter weight, and amounts of wastes and residues. Based on the technical utility of cereals, the crop is either directed to further processing by milling or to applications of feed industry. Oil plants may e.g. be used as raw materials for plant oil industry or for fuel production. A special requirement for malting barley is its grain size, and low protein content. In case the protein content of barley is unsuitable for malting, barley is used for feed or for the production of ethanol.

According to the second aspect of the invention, the environmental impact index reflecting the environmental impact of the production of the cultivated plant is provided by means of energy and carbon dioxide factors and documented appendant information for the crop yield, and by computation methods and systems. Thus, the environmental impact index is a product criterion that may be easily understood and compared, said product criterion containing the sum of the information on the environmental impact of the cultivation parameters selected by a specific farmer, and of the crop produced by said farmer on the basis of these parameters, and the information on the influence and significance of each parameter. The environmental impact index is preferably a numerical value defined by the ratio of output to input for the production of the cultivation plant. Said index may be understood as a tool provided by the method described above on the basis of the technical operations associated with the cultivation (appendant information, analysis of the crop), said index thus reflecting the conversion of energy thus achieved and produced carbon dioxide load for the environment, and being useful for the selection of cultivation parameters for the next season for modifying said assessment criterion to become more favourable.

The term input refers to the energy consumed during the life cycle of cultivated plant such as produced wheat, and carbon dioxide emission caused by the production. Energy consumption caused by cultivation procedures, and carbon dioxide emissions to the atmosphere during the cultivation, and possibly the nutrient balance particularly monitoring and measuring nutrient releases into surface waters are incorporated into the input.

The term output refers to the energy produced by or bound to the plants, and carbon dioxide consumption. Output thus comprises the energy value, the amount of bound carbon dioxide and any nutrients bound to the plant measured on the basis of crop analysis. The environmental impact preferably comprises the total energy balance and carbon dioxide balance for the production of the cultivation plants.

According to a preferable embodiment, the environmental impact index obtained is expressed as the sum of two numerical values X+Y, that is as the energy-carbon dioxide index where X and Y depend on each other through selected cultivation procedures. This means that the amount of energy produced is X times the amount of energy used for the production, and similarly, the amount of bound carbon dioxide is Y times the amount produced in cultivation. As already previously stated, the assessment of the total impact may be complicated since a certain production input affects both of these partial factors, e.g. both the energy consumption and carbon dioxide emission, and the effect on one of these factors may be weak or even opposite, while the effect on the other factor may be considerable, the total effect thus being the sum of these partial factors.

To increase or maximize the positive environmental impact obtained with the method of the invention, environmental impact index and determination thereof are utilized for the future cultivation of the coming years and for the planning thereof.

The farmer may utilize the environmental impact index according to the invention for enhancing the plant production by selecting operations with highest relative energy consumption and highest environmental carbon dioxide loads to be the targets for the improvement of the production of the next crop, said operations being provided by the method according to the invention as the final result of the computations. The environmental impact index and parameters with the highest effect thereon are supplied to the farmer to iteratively direct his choices for the future seasons, and to consumers to direct their choices towards consumer goods with favourable environmental impact. Using the environmental index of the invention, yield expressed as kg/hectare and commercial quality of the produced crop for the selected variety may easily be converted to an energy value and amount of CO₂ bound by the crop.

Typically, criteria are set by those who perform further processing to classify the crop on the basis of the environmental impact index. Thus, factors determining the environmental index may then be compared within one crop season between different farmers, or over several crop seasons by means of changes made by the same farmer. Understanding of the effects of these determining factors for the establishment of the environmental index is helpful for the selection of the cultivation parameters to attain the desired final result, and quality class according to the environmental impact criterion of the crop.

The effect of the parameters selected by the farmer on the environmental impact index may be monitored and alternative combinations to be assessed for possible use by the farmer for the next cultivation season may be provided on the basis of energy and carbon dioxide balance computations. It is thus possible to determine the final result for any parameters to be used or for attaining the desired index, preferably for the optimization of the combined result thereof.

An advantage for the farmers is the possibility to prove the environmental friendliness of their farming activity in comparison to other producers, their awareness of the environmental impact of the farming activities, and their readiness to influence this environmental impact. Moreover, procedures according to the invention provide concrete numerical values describing the quality of these activities.

According to the second aspect, the invention provides an environmental impact index determined by the method according to any of the claims 1-7.

The environmental impact index according to an embodiment of the invention may be used for the determination of the market value of a food product containing a cultivation plant, and the traceability thereof and/or for directing consumer decisions.

The environmental impact index provided may easily be applied in practice. With this method, for instance different plant species may be compared with each other. In practice, plant rotation defines for the most part the location of different cultivation plants on different field sections within the farm, and thus different plants are cultivated on the same section in different years. It is thus convenient to provide means for environmentally most friendly production on the same section in different years, which is made possible by an embodiment of the invention.

According to the third aspect, the invention provides a system for determining the environmental impact of cultivation plants and for increasing or maximizing a positive impact, said system comprising parts necessary for the implementation of the method. Said parts at least comprise a container such as a sack, bag, or pallet, for storing a representative sample of the crop, and means such a physical print on the sample container or paper form or a microchip containing the information or the like, for recording the appendant information; analysis equipment for assaying energy and carbon dioxide factors of the crop yield from the representative sample; central processing unit for calculations arranged for the determination of the environmental impact index on the basis of the energy and carbon dioxide data of the representative sample, given by the analysis equipment, and appendant information therefor; and referral for sending of the environmental impact index to information users.

Fourth aspect of the invention provides an apparatus for the determination of the environmental impacts of cultivation plants and for increasing or maximizing a positive environmental impact, said apparatus comprising at least an inlet for the reception of the appendant information; an analyzer for the analysis of energy and carbon dioxide factors of a representative sample of a crop; central processing unit for the determination of an environmental impact index from the representative sample on the basis of appendant information and energy and carbon dioxide factors; and an outlet for providing the environmental impact index.

The fifth aspect of the invention provides a software containing software codes arranged for the determination of the environmental impact of the production of cultivation plants and for increasing or maximizing a positive environmental impact. This software contains codes arranged to receive the appendant information; to receive data analyzed on the basis of the energy and carbon dioxide factors of a representative sample; to determine an environmental impact index on the basis of the appendant information and analysis data; and to provide the environmental impact index.

The invention is further illustrated with the following examples without limiting the invention thereto.

### EXAMPLES

### Example 1

Industry using wheat as starting material makes contracts with 611 wheat producing farmers for the provision of starting material for milling processes. The object is to take the environmental impacts of wheat production into consideration.

Production contracts made with the cereal purchase unit of the industry demands that said farmers record following production factors used for cultivation:
1. arts of tilling and fertilization of the field, and driving time of the tractor therefor (e.g. ploughing, light tilling or direct sowing)
2. amounts of nitrogen, phosphorus and potassium used for fertilization per hectare, and the amount of other soil improving agents per hectare
3. amounts of plant protecting agents (herbicides, fungicides, insecticides, and growth regulators, and the amount of tractor work load for plant protection
4. operations used for harvesting such as amount of combined harvester work, and amount of fuel oil for drying the crop
5. the amount of crop, conditioned to moisture content of 15%, per hectare
6. distance and art of transportation from the farm to the reception site of the crop indicated by the industrial user.

After harvesting, each wheat producer delivers a representative grain sample of their respective crop in a plastic bag to a cereal grain laboratory indicated by the industry, wherein the determination of the amounts of starch, protein and fats, and ash content of the crop, together with cultivation information on a prefilled form printed on the bag. Moreover, quality properties such as falling number, hectoliter weight, and amounts of wastes and residues reflecting the technical utility value of the crop are determined in the laboratory. On the basis of this technical utility value, the wheat yield is either directed for use in milling or for feed industry.

The energy produced by the crop per hectare (output) is calculated in the laboratory individually for each farmer, knowing that the energy value of one gram of starch, protein, and plant fat is 1.74 kJ, 2.34 kJ and 3.68 kJ, respectively. Moreover, the amount of carbon dioxide bound in the crop is calculated knowing that 1.47 kg of carbon dioxide is bound by the production of 1 kg of dry matter.

Thereafter, the amount of energy used for production, and total emissions of carbon dioxide (input) are calculated in the laboratory individually for each farmer based on the information provided by the farmer. Calculations are performed on the basis of the fuel amounts used for tractors, combined harvesters, transportation, and drying of the crop, knowing that the heat value of fuel oil is 43 MJ/kg, and CO₂ emission for one litre of fuel oil is 2.7 kg. In addition, the amount of energy consumed on the farm for fertilization and plant protection is determined, knowing that the amount of energy required for the production of one ton of nitrogen, phosphorus and potassium is 50 GJ, 12 GJ and 7 GJ, respectively. Moreover it is known that CO₂ emissions in the production of one kg of nitrogen, phosphorus and potassium are 5.3 kg, 0.2 kg, and 0.5 kg, respectively. Is is further known that the amount of energy required for the production of one kg of active plant protecting agent is about 0.36 GJ, and CO₂ emissions in the production of pesticides are 22 kg for each produced kg of the active agent.

Calculations are carried out using software designed for this purpose. Energy and carbon dioxide indices are expressed as the input/output ratio. Energy-carbon dioxide index is 7+5 for the crop that is environmentally most favourable, meaning that the amount of energy produced is 7 times higher than the amount of energy used for wheat production, and similarly, the amount of carbon dioxide bound by the wheat yield is 5 times higher that emissions due to the production.

Results of the calculation are given to each farmer, while the cereal purchase unit of the industry receives a summary of the results. Each result given to the farmer contains a median as a reference value, the poorest quarter and the best quarter. The starting material produced is directed by the cereal purchase unit either to milling or to feed processing according to utility value of the crop. Products of the milling industry that are environmentally most favourable are provided with a label of the energy-carbon dioxide index, and thus a higher market value is attained for these products than for those without such a label, produced using wheat with a lower energy-carbon dioxide index.

Improvement plan for each voluntary wheat producer is made by the cereal purchase unit for the next cultivation season, enabling the modification of the index values towards more favourable environmental impact.

### Example 2

The farmer may use the environmental impact index acquired according to Example 1 for enhancing plant production by selecting operations with highest relative energy consumption and highest environmental carbon dioxide loads to be the targets for the improvement of the production of the next crop, said operations being provided by the final results of the calculations according to Example 1.

The farmer utilizes the environmental impact index acquired for enhancing plant production by selecting the modification of nitrogen fertilization for energy consumption, and modification of spraying of plant protecting agents for carbon dioxide load to be the targets for the improvement of the production of the next crop.

The farmer knows that similar or, relatively speaking, equal energy consumptions and carbon dioxide emissions are not necessarily caused by spraying of plant protecting agents in comparison to other production inputs or methods such as art of fertilization. In case spraying of plant protecting agents is selected as target of improvement, energy consumption calculated per field hectare may be very low, while CO₂ emissions caused by the same production input may be very high. There are various alternatives for the reduction of the CO₂ emissions of herbicide spraying: as herbicides, instead of phenoxy acids applied in amounts of few litres per hectare, and causing high CO₂ emissions, low dose products based on sulphonyl urea used only in low amounts of few grams per hectare, may be selected according to the selected cultivation plant, weed species present in the field, and protection efficiency needed.

Next spring, the farmer fertilizes the fields only with phosphorus and potassium, without using any nitrogen fertilizers. A mixture of peas and wheat is sown by the farmer, peas binding atmospheric nitrogen to the soil for plants. Due to the cultivated variety, phenoxy acid is selected to be the herbicide, used only half of the amount recommended by the manufacturer with a single spraying. Herbicidal control efficiency of the half dose of the product for the mixed culture of wheat and peas is as high as that of the whole herbicide dose for pure wheat cultivation. This mixed cultivation requires no fungicides.

For the farmer, the environmental index is improved by 4% with respect to energy consumption, and 5% with respect to carbon dioxide emissions. Moreover, the amount of unutilized nutrients remaining in the soil is 50 kg lower than in previous years according to nutrient balance calculations.

### Example 3

The environmental impact index may be significantly improved by the selection of the variety of the cultivated plant species in case a variety reliably giving high yields is selected by the farmer for the production. A variety with high yields binds more carbon dioxide than a variety with low yields. Energy value of a variety with high yields is higher than that of varieties with low yields. Under local cultivation conditions, a variety with high yields should ripen early enough during the cultivation period. Crop yields and growing times are strongly and positively related. It is thus not easy to find an early variety with high yields, having a favourable influence on environmental index. By means of the environmental index, the amount of the yield by a potential wheat variety on the variety list in kg/hectare and commercial quality thereof are converted into energy values and amounts of CO₂ bound in the crop.

Due to the location of the farm, the farmer is only able to cultivate varieties having a cultivation time of up to 102 days. Varieties having most suitable energy values and amounts of CO₂ bound by the crop are selected by the farmer from the variety list on the basis on the environmental index. Particularly, since the energy component of the environmental index should be improved by the farmer, the amount of energy in the crop is weighed for the selection among otherwise equivalent varieties.

Due to the selection of a novel variety, the environmental index of the farmer was improved by 6% for energy value and 3% for carbon dioxide value.

### Example 4

Information acquired with the environmental index may be utilized for better detection of slow changes.

Improved resistance to diseases of the novel variety is significant for the farmer influencing the environmental index. In case the the variety is resistant to several plant diseases present in the region, the need for fungicides decreases. This particularly reduces CO₂ emissions, influencing the environmental index of the first cultivation season.

In the next cultivation season following the introduction of the novel wheat variety, the environmental index for the farmer is further improved with respect to both the energy and CO₂ values. However, before long, the resistance to diseases of the varieties will collaps, causing an unnoticed increase in necessary fungicide use in the form of both spraying frequencies and amounts sprayed, or more efficient novel agents will be selected for spraying. The environmental index will reveal this systematic change before the farmer necessarily pays any attention to it. The change is shown by a modest but evolving trend in several parameters, suggesting that something should be done to prevent the next cultivation season from being worse than the previous one.

In the third year, the positive trend comes to an end notwithstanding the fact that energy consumption due to machine work load is improved by the farmer during the past cultivation season. More precice analysis of the environmental impact index shows that the resistance to diseases of the cultivated varieties is collapsing. It has been necessary to use little higher amounts of active agents during the summer for fungicide spraying even though the prevalence of pathogenic agents is the same as in previous years. Also the quantity of the yield is somewhat lower than in previous years.

Now it is time to change the variety again to obtain a positive environmental output for the next crop, and to avoid unnecessary spraying of plant protecting agents to the fields.

### Example 5

Monitoring of the environmental impact index may also be utilized in graindrying.

High amounts of energy may be consumed in drying of crop yield, especially in case the cultivation season has been rainy and wet during harvesting. Energy consumption indicated by the environmental impact index may at best be significantly lowered in case the cultivations are subjected to late glyphosate spraying causing forced ripening of the plants and lowering the moisture content of the grains by some percents. For the environmental impact, it is very challenging to find a balance between the environmental impact index improved by the fuel oil savings and the environmental impact index decreased on the other hand by use of plant protecting agents such as glyphosate and application work load associated therewith.

Harvesting time of the crop approaches, and according to the weather forecast, harvesting period will be bad. Three weeks prior to threshing, the farmer, however, decides to perform glyphosate spraying of the wheat cultivations because of the rainy weather predicted. The decision for spraying is also supported by the fact that the weed Elymus repens, is found more than normally in the fields. Its presence in the crop would still increase the drying costs. Late glyphosate spraying also eradicates permanent weeds of the field. After harvesting, the protecting efficiency would probably be considerably lower and demand use of higher glyphosate doses per hectare or possibly one or several additional treatments instead of this single treatment in the harvesting step. The environmental impact index also indicates the influencing trend of the treatments in different harvesting steps.

Considering energy and CO₂ values, the environmental impact index is improved by 2% by correctly timed measures.

### Example 6

The environmental impact index may also be utilized for the comparison of the correct timing of fertilization, and efficiency of targeted fertilization.

The environmental impact index of the field may be improved by the selection of a proper fertilization strategy, according to the cultivated plant and variety, soil type and soil inherent nutrient content and expected yield potential obtainable for these factors. The types and amounts of nutrients to be added to the field are selected by the farmer. Moreover, the farmer decides whether the nutrients are introduced for the targeted crop all at once as the so-called multinutrient fertilizer, which is applied in a single driving operation, and simultaneously with the sowing of the seeds, or distributed in single nutrient fertilizers separately during the cultivation season according to the development of the crop probably resulting in a significant increase of the crop volume due to more precice dosage. Single nutrient fertilizing increases the driving frequencies of tractors to two or three. Targeted crop yield for the multiple nutrient fertilizer is not necessarily attained, for instance due to dry summer, and therefore, decreased crop yield may finally lower the environmental impact index considerably, even though the purpose of the decreased tractor work load was originally the opposite.

Previously, the farmer has only used multinutrient fertilizers with high nitrogen content for fertilization. The fertilization is carried out in association with sowing in the form of the so-called placement fertilization where the fertilizers are placed between seed rows. To lower the resulting environmental impact index, the farmer decides in the next year to use only multinutrient fertilizers with low nitrogen content for the placement fertilization, and to place the multiple nutrient fertilizers in the same row with the seeds to boost the efficiency. During the cultivation season, additional nitrogen is applied in the form of ammonium nitrate according to the crop yield potential, using top dressing.

As a result of the boosting of the fertilization efficiency, the crop volume increases by 9% in comparison to the average volume attained in the previous years. The effect of this on the environmental impact index is also significant: also the energy and CO₂ values are improved by nearly 6%, and 8%, respectively. Moreover, the nutrient balance of the wheat fields are seen to be improved on an average by 4%.

### Example 7

Wheat producers A, B and C make production contracts with the industry using wheat. The contract requires the farmers to collect necessary production data for calculation of the energy-carbon dioxide index for the production. Wheat production is carried out as described in Example 1, recording the production factors used for cultivation, and sending the data with a representative sample of the wheat crop to a cereal grain laboratory as agreed with the industry.

The samples are analyzed as described in Example 1 in the laboratory. Moreover, the laboratory carries out calculations of the energy-carbon dioxide index with a software designed for this purpose, to give a report specific for each farmer indicating the targets for improvement, and a reference index for comparison with other farmers, the software also sending this information to the farmers and cereal purchase unit.

The aim set by the cereal purchase unit is to acquire wheat for milling having an energy-carbon dioxide index of 7+5, that is, the amount of energy produced is 7 times higher than that used for wheat production, and the amount of bound carbon dioxide is 5 times higher that emissions due to used production techniques.

Wheat lots of the farmers B and C are chosen by the cereal purchase unit to be received by the milling unit of the company for further processing due to superior technical quality of starch and protein of the wheat lots, based on utility value analysis, and further, the lots are produced according to the energy-carbon dioxide index set as the target. Raw material produced by the farmer A is directed to feed industry due to the energy-carbon dioxide index of the material of 4+4, the falling number indicating that the quality of starch in the lot is also too low for milling purposes.

The cereal grain laboratory delivers this result to the farmers in the form of a report classifying each wheat producer according to an index, and showing the factors most significant for the index. Results of the computations are discussed by the cereal purchase unit together with the farmers A, B and C, comparing the production factors used on each farm with one another to suggest changes, and to increase production with most favourable environmental impact in the future.

There is a significant difference in fuel oil consumption between the farms. Careful study of the report reveals that there are no differences in the amounts of energy used for transportation, work load of combined harvesters, or crop drying. Only the farmer A has decided to reduce costs for plant protection agents. Therefore, also work load for tractors is lower for these operations than for the farmers B and C.

Greatest difference in fuel costs between the farms is caused by soil tilling procedures. Farmer A has ploughed his wheat fields in autumn and harrowed the fields once in spring, followed by sowing wheat seeds into the field. Farmer B has lightly tilled the fields once in autumn and once in spring, followed by sowing the seeds. Farmer C has used the direct sowing technology that is his fields were non-tilled until sowing the seeds.

As a result of the choice of these working techniques, total energy consumption for farmer A expressed as fuel costs is about 15% higher than for farmer B, and 24% higher than for farmer C. Also as a result of the choice of these working techniques, carbon dioxide emissions for farmer A are 12% higher than for farmer B and 23% higher than for farmer C. To change the situation, readiness to adopt lighter tilling techniques used in the production chain should be increased on the farm of farmer A.

Farm A having tried to reduce the costs associated with the use of plant protecting agents, plant diseases have caused crop losses due to unfavourable cultivation conditions. Farmer A decides to focus on this problem by starting to follow more carefully prediction services in this field and to use necessary plant protection solutions. For the next season, crop with high yield, and at the same time the commercial quality thereof as required by the milling industry for starch and protein are thus assured.

Even though grain yield produced by farmer B is by 500 kg/hectare higher than that of farmer C, the report obtained suggests that in spite of the superior crop, farmer B could further improve the efficiency of fertilization, and reduce fuel consumption. Farmer B also noticed that carbon dioxide emissions may be reduced by proper choice of the plant protecting agents.

Table 1 shows the energy-carbon dioxide indices for wheat production of the farmers A, B and C. Crop volumes for farmers A, B and C were 3000, 5500 and 5000 kg/ha, respectively.

TABLE 1

| | Farmer | | | | | |
|---|---|---|---|---|---|---|
| | A | | B | | C | |
| | Energy | CO₂ | Energy | CO₂ | Energy | CO₂ |
| | GJ/ha | kg/ha | GJ/ha | kg/ha | GJ/ha | kg/ha |
| Grain yield | 47.7 | 3837 | 87.5 | 7034 | 79.5 | 6395 |
| Fuel costs, | 4.7 | 352 | 4.1 | 313 | 3.8 | 287 |
| total | | | | | | |
| Fertilization | 6.6 | 659 | 7.1 | 709 | 5.3 | 534 |
| and soil tilling | | | | | | |
| Plant | — | — | 0.1 | 351 | 0.1 | 346 |
| protection | | | | | | |
| Production | 11.3 | 1011 | 11.3 | 1373 | 9.2 | 1167 |
| input, total | | | | | | |
| Energy-carbon | 4.2 | 3.8 | 7.7 | 5.1 | 8.6 | 5.5 |
| dioxide index | | | | | | |

### Example 8

Wheat and rye with a favourable environmental impacts for milling purposes, and barley with a favourable environmental impact for malting processes are produced by farmers under contract for the industry. According to the contract, farmers should record necessary appendant information to enable the industry to calculate the influence thereof on the energy-carbon dioxide index.

Production data from 985 wheat fields, 92 rye fields and 1119 barley fields are gathered in the system. In the production of wheat, rye and barley, mean energy-carbon dioxide index is 6+4, meaning that the energy value of the crop is 6 times higher than the energy consumed by the production thereof, and further, the amount of carbon dioxide bound in the crop is 4 times higher than the carbon dioxid emissions of the inputs used for the production. The poorest quarter is only able to double or triple the numerical index reflecting the energy-carbon dioxide balance of the production.

The aim of the company is to use only cereals having an energy-carbon dioxide index of at least 7+5 as starting material for milling and malting processes. In the year of the agreed production contract, the best quarter exceeds this limit value. More careful analysis of the results shows that the best quarter is able to improve the energy-carbon dioxide index specifically by using lighter soil tilling operations, and by achieving clearly higher crop responses for the fertilization input. On the other hand, the carbon dioxide balance may be increased by efficient, adequate and correctly timed use of plant protection agents. Healthy crops are able to utilize nutrients more efficiently, and more carbon dioxide is bound by the crop. The best quarter is able to achieve better crop volumes per hectare, the increase being 400 to 500 kg/ha compared to average crop volumes of all farmers.

On the basis of the analysis, the industry decides to set as a realistic middle term goal to develop the energy-carbon dioxide index for enabling the best quarter of the farmers to achieve an energy-carbon dioxide index of 9+7, and an index of 10+8 as a the long term goal.

Table 2 shows the energy-carbon dioxide indices for wheat, rye and barley produced according to a contract

TABLE 2

| | Plant species | | | | | |
|---|---|---|---|---|---|---|
| | Wheat | | Rye | | Barley | |
| | Energy | CO₂ | Energy | CO₂ | Energy | CO₂ |
| | index | index | index | index | index | index |
| Lowest value | 2.0 | 1.2 | 4.0 | 2.8 | 1.6 | 1.3 |
| Poorest quarter | <4.7 | <3.9 | <5.6 | <3.7 | <5.9 | <4.4 |
| (25^{th} percentile) | | | | | | |
| Median value | 6.3 | 4.2 | 6.1 | 4.2 | 6.3 | 4.6 |
| (50^{th} percentile) | | | | | | |
| Best quarter | >6.8 | >4.7 | >7.0 | >4.7 | >6.8 | >5.0 |
| (75^{th} percentile) | | | | | | |
| Best value | 15.2 | 12.6 | 12.7 | 7.9 | 11.8 | 12.3 |

### Example 9

A farmer has made a contract on the production of oat grains with the industry. The contract demands the farmer to record the cultivation data for the production to enable the industry to calculate the energy-carbon dioxide index. Oat grain production is carried out by the farmer as described in Example 1, the farmer also recording the production factors used for the cultivation and delivering this information with a representative oat seed sample to the cereal laboratory as agreed.

Cereal laboratory analyses the samples as described in Example 1. In addition, calculation of the energy-carbon dioxide index is performed by the laboratory using a software designed for this purpose, said software being also used for the comparison of the data of the farmer with the median value obtained from all producers of oat grains, and finally for sending the information to the farmer and cereal purchase unit.

The report received by the farmer shows that the energy production is lower by 16.5 GJ/haand the amount of bound carbon dioxide is lower by 2577 kg/ha than the median value obtained from all producers of oat grains. This median value is obtained from producers of oat grains using lighter soil tilling techniques more efficiently by 0.4 GJ/ha and with carbon dioxide emissions that are 13 kg lower than those of the farmer. Higher CO₂ emissions are caused by nitrogen fertilization and weed control for the farmer compared to the median values for oat producers. The highest reduction of the energy balance for the farmer is caused by nitrogen and phosphorus fertilization as well as choices relating to the use of plant protection agents.

Table 3 shows the energy-carbon dioxide index for the production of oat grains for the farmer, and the deviation of the input factors from the median value in the same group.

TABLE 3

| | | | | | Lower than | |
|---|---|---|---|---|---|---|
| | Farmer | | Group median | | median | |
| | Energy | CO₂ | Energy | CO₂ | Energy | CO₂ |
| | GJ/ha | kg/ha | GJ/ha | kg/ha | GJ/ha | kg/ha |
| Grain crop | 65.0 | 3837 | 81.5 | 6414 | −16.5 | −2577 |
| Fuel costs, | 4.8 | 352 | 5.0 | 395 | | |
| total | | | | | | |
| reducedtilling | 1.3 | 93 | 0.9 | 80 | −0.4 | −13 |
| plant protection | 0.3 | 19 | 0.3 | 19 | | |
| work load | | | | | | |
| combined | 0.6 | 49 | 0.6 | 56 | | |
| harvester | | | | | | |
| work load | | | | | | |
| drying of | 191 | 3.2 | 239 | | | |
| grain 2.6 | | | | | | |
| transportation | 0.003 | 0.24 | 0.006 | 0.47 | | |
| to the factory | | | | | | |
| 40 km | | | | | | |
| Fertilization | 6.5 | 659 | 5.7 | 556 | −0.8 | −103 |
| and soil | | | | | | |
| improvement | | | | | | |
| liming 0.05 | 3 | 0.10 | 5 | | | |
| nitrogen | 5.9 | 624 | 4.7 | 508 | −1.2 | −116 |
| fertilization | | | | | | |
| phosphorus | 0.1 | 3 | 0.2 | 4 | −0.1 | −1 |
| fertilization | | | | | | |
| potassium | 0.4 | 29 | 0.6 | 39 | | |
| fertilization | | | | | | |
| Plant protec- | 0.2 | 327 | 0.1 | 327 | −0.1 | |
| tion, total | | | | | | |
| weed control | 0.1 | 215 | 0.05 | 166 | −0.05 | −49 |
| spraying | | | | | | |
| disease con- | 0.04 | 66 | 0.03 | 88 | −0.01 | |
| trol spraying | | | | | | |
| growth | 0.06 | 46 | 0.02 | 73 | −0.04 | |
| regulators | | | | | | |
| Production | 11.5 | 1338 | 10.7 | 1278 | −0.8 | −60 |
| inputs, total | | | | | | |
| Energy-carbon | 5.6 | 2.9 | 7.6 | 5.0 | −2.0- | −2.1 |
| dioxide index | | | | | | |

While the above description comprises several details, they are only presented for the illustration of the invention, without wishing to limit the scope thereof. Presented details may be combined in many ways in various aspects and embodiments of the invention. For those skilled in the art, it is obvious that the solutions presented in the invention may be altered or modified within the scope of the inventive solution.

1. Method for determining the environmental impact of a production of cultivation plants and for increasing or maximizing a positive environmental impact, characterized in that
a. production parameters for cultivation are selected and cultivation procedures are performed by using a low assumed input with respect to output, and further, the performed procedures are documented to give appendant information,
b. crop yield thus produced is harvested,
c. a representative sample of the crop yield is delivered to reception analysis with the appendant information,
d. energy and carbon dioxide factors of the crop yield are analysed using the representative sample,
e. environmental impact index reflecting the environmental impact of the production of said cultivation plant is determined on the basis of the energy and carbon dioxide factors of the crop yield and the appendant information, and
f. said environmental impact index and the determination thereof is utilized for the production of cultivation plants in the next cultivation season.

2. Method according to claim 1, characterized in that said cultivation plant is selected from the group consisting of oil plants, preferably turnip rape, oil seed rape, Camelina, sunflower or soybean; cereals for human and animal nutrition, preferably wheat, rye, barley or oat; leguminous plants, preferably peas or broad bean; potato, corn and sugar beet.

3. Method according to claim 1 or 2, characterized in that said production parameters of the cultivation and cultivation procedures comprise information on soil tilling, more preferably information on soil tilling procedures and dates; information on fertilization, more preferably information on fertilizers used, used amounts of fertilizers and frequency of application; information on the use of machines and apparatuses, more preferably information on fuel consumption for said machines and times of use; information on plant protection, more preferably information on used agents, amounts and frequency of applications thereof; and harvesting information, more preferably information on crop quality and amount, way of harvesting, subsequent treatment, transportation distance and means of transportation.

4. Method according to claim 1, characterized in that said appendant information are in the form of a prefilled form, preferably attached to the representative sample of the crop yield.

5. Method according to claim 1, characterized in that said energy factors of the crop yield comprise the amounts of starch, protein and fat, ash content and qualitative features relevant to technical utility, preferably falling number, hectoliter weight, and amounts of wastes and residues.

6. Method according to claim 1, characterized in that said environmental impact comprises the total energy and carbon dioxide balances associated with the production of cultivation plants.

7. Method according to claim 1, characterized in that said environmental impact index is a numerical value defined as the ratio of input to output of the production of a cultivation plant.

8. Environmental impact index provided by the method according to claim 1.

9. Use of the environmental impact index according to claim 8 for the determination of the market value of a food product containing a cultivation plant, and the traceability thereof and/or for directing consumer decisions.

10. System for determining the environmental impact of a production of cultivation plants and for increasing or maximizing a positive environmental impact, characterized in that said system comprises
a. an appendant information form, a container for storing a representative sample of the crop, and means for storing production parameters of the cultivation,
b. analysis equipment for assaying energy and carbon dioxide factors of the crop yield from the representative sample,
c. central processing unit for calculations arranged for the determination of the environmental impact index on the basis of the energy and carbon dioxide data of the representative sample, given by the analysis equipment, and appendant information therefore; and
d. referral for sending the environmental impact index to information users.

11. Apparatus for determining the environmental impact of a production of cultivation plants and for increasing or maximizing a positive environmental impact, characterized in that it comprises
a. an inlet for the reception of the appendant information;
b. an analyzer for the analysis of energy and carbon dioxide factors of a crop yield;
c. central processing unit for the determination of an environmental impact index on the basis of appendant information and energy and carbon dioxide factors; and
d. an outlet for providing the environmental impact index.

12. Software containing software codes arranged for the determination of the environmental impact of the production of cultivation plants and for increasing or maximizing a positive environmental impact, characterized in that said software contains codes arranged:
a. to receive appendant information;
b. to receive analyzed data about the energy and carbon dioxide factors of a sample of a crop yield
c. to determine an environmental impact index on the basis of the appendant information and analysis data; and
d. to provide the environmental impact index.
